(19) United States
(12) Patent Application Publication
(10) Pub. No.: US 2005/0215812 A1
(43) Pub. Date: Sep. 29, 2005

(54) Processes for the preparation of iodinated amino-aryl compounds

(75) Inventor: Ronald S. Michalak, Congers, NY (US)

Correspondence Address: COZEN O' CONNOR, P. C., 1900 MARKET STREET, PHILADELPHIA, PA, 19103-3508, US

(73) Assignee: Wyeth, Madison, NJ (US)

(21) Appl. No.: 11/088,568

(22) Filed: Mar. 24, 2005

Related U.S. Application Data
(60) Provisional application No. 60/557,014, filed on Mar. 26, 2004.

(51) Int. Cl. C07C 253/30; C07C 229/68

(52) U.S. Cl. 558/410; 564/442; 562/456

(57) ABSTRACT

The present invention provides processes for the preparation of amino-aryl iodides wherein a micronized amino-aryl compound is reacted with an iodinating reagent.

### CROSS REFERENCE TO RELATED APPLICATION

This application claims priority benefit of U.S. Provisional Application Ser. No. 60/557,014 filed Mar. 26, 2004, which is incorporated by reference herein in its entirety.

### FIELD OF THE INVENTION

The present invention relates to processes for the high yielding production of iodinated aryl amines wherein an aryl amine of reduced particle size is reacted with an iodinating agent.

### BACKGROUND OF THE INVENTION

Iodinated amino-aryl compounds have considerable value as synthetic intermediates for a wide range of substances useful in various industrial settings including the pharmaceutical industry. For example, 2-iodoanilines are known precursors useful in the synthesis of a large number of indoles, including 2,3-disubstituted indoles, some with reported utility as potential migraine headache drugs. Larock, R. C., Yum, E. K., Refyik, M. D., J. Org. Chem., 1998, 63, 7652, and references cited therein. A number of methods for the iodination of amino-aryl compounds have been reported in the literature but they either result in low to moderate yields or require the use of expensive reagents that require separate preparation. For example, the mono-iodination of 4-chloro aniline shown in Scheme I has been described in the literature, but the most commonly reported methods of adding molecular iodine to an aqueous solution of sodium bicarbonate, Xiao, W.; Alper, H. J. Org. Chem. 1999, 64, 9646-9652; Callaghan, P. D.; Gibson, M. S.; J. Chem. Soc. (C), 1970, 2106-2111, or calcium carbonate, Dains, F. B.; Vaughn, T. H.; Janney, W. M., J. Am. Chem. Soc., 1918, 40, 932; Breukink, K. W.; Krol, L. H.; Verkade, P. E.; Wepster, B. M., Rec. Trav Chim. Pay-Bas, 1957, 76, 401; Rosowsky, A.; Marini, J. L.; Nadel, M. E.; Modesi, E. J. J. Med. Chem., 1970, 13, 882, result in only moderate reported yields of 53% to 69%. Higher yields have been achieved with specially developed reagents such as benzyltrimethylammonium dichloroiodate (BnNMe₃ICl₂, 86%), Kajigaeshi, S., Kakinami, T.; Yamasaki, H.; Fujisaki, S.; Okamoto, T. Bull. Chem. Soc. Japan 1988, 61, 600-602 and bis(pyridine)iodonium(I)tetrafluoroborate (Ipy2BF4, 99%). Ezquerra, J.; Concepcion, L.; Barluenga, J.; Perez, M. J. Org. Chem. 1996, 61, 5804-5812. 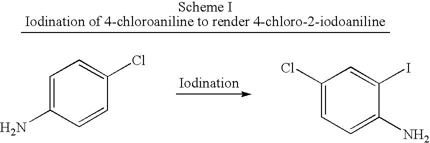
Accordingly, improved synthetic routes to iodinated aryl amines such as 4-chloro-2-iodoaniline are needed.

### SUMMARY OF THE INVENTION

In some embodiments, the present invention provides processes for the preparation of a compound of Formula I: 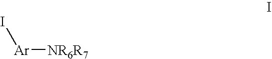
wherein:
Ar is a mono-, bi- or tricyclic aryl or heteroaryl ring system optionally containing up to four substituents independently selected from the group consisting of halogen, C₁₋₆ alkyl, CN, NO₂, CHO, COC₁₋₆ alkyl, CO₂H, CO₂C₁₋₆ alkyl, C₁₋₆ alkoxy, phenyl and C₁₋₆ thioalkyl, wherein the phenyl can be optionally substituted with from 1 to 3 substituents independently selected from the group consisting of C₁₋₃ alkyl, halogen, C₁₋₃ alkoxy, CN, NO₂, CHO, and phenyl;
R₆ and R₇ are each independently selected from the group consisting of H, C₁₋₆ alkyl and a nitrogen protecting group; comprising:
reacting an amino aryl compound of Formula II: 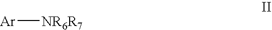
with an iodinating agent, wherein the amino aryl compound of Formula II has an average particle size of about 100 μm or less.

In some preferred embodiments, the compound of Formula II is an aniline of Formula III: 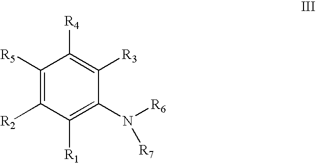
wherein R₅ is selected from the group consisting of halogen, C₁₋₆ alkyl, CN, NO₂, —CHO, COC₁₋₆ alkyl, CO₂H, CO₂C₁₋₆ alkyl, C₁₋₆ alkoxy, phenyl and C₁₋₆ thioalkyl, wherein the phenyl can be optionally substituted with from 1 to 3 substituents selected from the group consisting of C₁₋₃ alkyl, halogen, C₁₋₃ alkoxy, CN, NO₂, CHO, and phenyl; and
R₁, R₂, R₃ and R₄ are each independently selected from the group consisting of hydrogen, halogen, C₁₋₆ alkyl, CN, NO₂, CHO, COC₁₋₆ alkyl, CO₂H, CO₂C₁₋₆ alkyl, C₁₋₆ alkoxy, C₁₋₆ thioalkyl and phenyl, wherein the phenyl can be optionally substituted with from 1 to 3 substituents selected from the group consisting of C₁₋₃ alkyl, halogen, C₁₋₃ alkoxy, CN, NO₂, CHO and phenyl;
provided that at least one of R₁ and R₃ is H.

In some embodiments, R₅ is halogen or C₁₋₆ alkyl, preferably halogen, and R₁, R₂, R₃, R₄, R₆ and R₇ are each hydrogen.

In some preferred embodiments, the compound of Formula II is 4-chloroaniline, and the reaction of the 4-chloroaniline with an iodinating agent is performed under conditions effective to form 2-iodo-4-chloroaniline.

In some embodiments of the foregoing, the compound of Formula II has an average particle size of less than about 80 μm, or an average particle size of less than about 60 μm, or an average particle size of less than or equal to about 50 μm, or an average particle size of less than or equal to about 40 μm. A typical lower limit is about 30 μm or less, e.g., 10 μm. In some preferred embodiments, the compound of Formula II has an average particle size of between about 30 μm and about 60 μm, or between about 40 μm and about 50 μm.

In some embodiments, the iodinating agent is molecular iodine. In further embodiments, the reaction of the amino aryl compound of Formula II and the iodinating agent is performed in the presence of a group I or group II metal iodide, preferably potassium iodide.

In some embodiments, the reaction of the amino aryl compound of Formula II and the iodinating agent is performed in an aqueous solution, preferably buffered with a weak base, preferably buffered with NaHCO₃.

In some embodiments, the reaction of the amino aryl compound of Formula II and the iodinating agent is performed in an aqueous solution comprising molecular iodine, or a group I or group II metal iodide, or both molecular iodine and a group I or group II metal iodide. Preferably, the metal iodide is potassium iodide. Preferably, the aqueous solution is buffered with a weak base, preferably NaHCO₃.

In some embodiments, the potassium iodide, molecular iodine, or both are added to a mixture of the amino aryl compound of Formula II, NaHCO₃ and water. In further embodiments, the potassium iodide and molecular iodine are added as an aqueous solution to a mixture of the amino aryl compound of Formula II, NaHCO₃ and water.

In some embodiments, the molecular iodine is employed in an amount of from about 1 to about 1.5 equivalents relative to the amino aryl compound of Formula II. In further embodiments, the processes further comprise the step of adding an inorganic reducing agent to the mixture subsequent to iodine addition and prior to product isolation. Preferably, the inorganic reducing agent is a group I or II thiosulfate, a group I or II metal sulfite or a group I or II metal bisulfite, preferably sodium thiosulfate.

In some embodiments, the compound of Formula I is isolated by filtration. In further embodiments, the filtered compound of Formula I is washed with a solvent, preferably water.

### DETAILED DESCRIPTION OF THE INVENTION

The present invention provides methods for the highly efficient production of iodinated amino-aryl compounds. The processes described herein utilize micronized amino-aryl compounds as efficient iodination substrates for the preparation of iodinated amino-aryl compounds. The term micronized as used herein refers to the use of amino-aryl compounds having an average particle size of less than 1 mm, preferably less than about 100 μm, more preferably less than about 80 μm, more preferably less than about 60 μm, more preferably less than or equal to about 50 μm, or less than or equal to about 40 μm. In some preferred embodiments, the amino-aryl compounds have an average particle size of between about 30 μm and about 60 μm, or between about 40 μm and about 50 μm.

It has been found in accordance with the present invention that use of such micronized amino-aryl compounds confers significant advantages in the iodination reaction in terms of providing a product of high purity and yield, without the use of more expensive reagents.

Micronization of the amino-aryl derivatives may be accomplished by a variety of physical techniques well known to those of skill in the art, including but not limited to milling (ball milling, attrition milling, and variants of these processes), microfluidization, spray drying or extrusion followed by exposure to a supercritical fluid. As used herein, the term “average particle size” means the average size of the particles of the amino-aryl starting materials as determined by any of the standard techniques known in the art. In some preferred embodiments, the particle size is determined by microscopic observation, sieving, or by light scattering, using standard instrumentation, for example and without limitation, a Mastersizer S Particle Size Analyzer, available from Malvern Instruments (Southborough, Mass.).

The iodination reactions described herein are preferably performed on aryl substrates possessing an amine functionality directly connected to an aryl group. As will be recognized by those of skill in the art, the regiochemistry and stoichiometry of the iodination is determined by several factors, including the positions available for substitution, the particular aryl ring being iodinated, the presence of other activating/deactivating groups, the solvent, reaction time and temperature, the number of iodinating equivalents used, etc. The overall reaction is shown below in Scheme II.

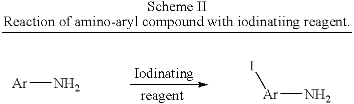

As used herein, the term “aryl”, employed alone or in combination with other terms, is defined herein, unless otherwise stated, as an aromatic hydrocarbon of up to 14 carbon atoms, e.g., 6-14 carbon atoms, which can be a single ring (monocyclic) or multiple rings (bicyclic, up to three rings) fused together or linked covalently. Examples of aryl moieties include, but are not limited to, chemical groups such as phenyl, 1-naphthyl, 2-naphthyl, dihydronaphthyl, tetrahydronaphthyl, biphenyl, anthryl, phenanthryl, fluorenyl, indanyl, biphenylenyl, acenaphthenyl, acenaphthylenyl, and the like. In some embodiments, the aryl moiety can be optionally substituted with from 1 to 4 substituents selected from the group consisting of halogen, C₁₋₆ alkyl, CN, NO₂, CHO, COC₁₋₆ alkyl, CO₂H, CO₂C₁₋₆ alkyl, C₁₋₆ alkoxy, C₁₋₆ thioalkyl and phenyl optionally substituted with from 1 to 3 substituents selected from C₁₋₃ alkyl, halogen, C₁₋₃ alkoxy, CN, NO₂, CHO and unsubstituted phenyl.

As used herein, the term “heteroaryl” denotes an aryl group as defined above, i.e., of up to 14 ring atoms, e.g., 5-14 ring atoms, that contains at least one non-carbon (“hetero”) ring atom. Preferably, heteroaryl groups contain from one to four such heteroatoms, preferably selected from one or more of O, N and S.

The micronized amino-aryl compounds can be reacted with a number of different iodinating reagents in accordance with the present methods to achieve the desired results. In general, iodinating reagents are reagents that are capable of donating an iodine atom to a target substrate. Useful iodination reagents or procedures include molecular iodine with or without the addition of an oxidant, a metallic salt or alumina; metal iodide salts together with oxidants; iodomercuration; electrochemical iodination; iodoamides; iodonium salts or transiodination procedures. In some preferred embodiments, micronized amino-aryl compounds are reacted with molecular iodine to form iodinated amino-aryl compounds. In some such embodiments, micronized amino-aryl compounds react with molecular iodine in the presence of a group I or group II metal iodide salt such as LiI, NaI, KI, CaI₂, and the like. In some preferred embodiments, the metal iodide salt is KI.

The reaction between the amino-aryl compound and the iodinating reagent can be performed by any of a variety of protocols known in the art, for example, by introducing the iodinating reagent to a mixture of the amino-aryl compound in an appropriate solvent, or by adding the amino-aryl compound to the iodinating reagent in such a solvent.

It is not necessary that the amino-aryl compound or the iodinating reagent be completely soluble in the solvent. Thus, in some embodiments, the amino-aryl compound will possess limited solubility in the solvent, and in other embodiments the iodinating reagent will possess limited solubility in the reacting solvent.

The solvent used for reacting the amino-aryl compound and the iodinating reagent can consist of a single solvent, or can be a mixture of two or more solvents. Where the reaction mixture includes two or more solvents, the mixture can be homogenous or heterogeneous.

The reactions of the processes described herein can be carried out in suitable solvents, which can be readily selected by one of skill in the art of organic synthesis. Suitable solvents include organic solvents, aqueous solvents, and combinations thereof. Suitable solvents are preferably substantially nonreactive with the starting materials (reactants), the intermediates, and/or products of the reaction at the temperatures at which the reactions are carried out, which can be any suitable temperature from the solvent's freezing temperature to the solvent's boiling temperature. Suitable organic solvents include, but are not limited to, hydrocarbons and halohydrocarbons, including pentanes, hexanes, heptanes, benzene, methylene chloride, chloroform, carbon tetrachloride, dichloroethane, toluene, mesitylene, chlorobenzene, polychlorobenzenes, bromobenzene, and the like; alcohols, including methanol, ethanol, propanol, isopropanol, butanol, sec-butanol, and the like; amides, including dimethylformamide, diethylformamide, acetamide, dimethylacetamide, and the like; ketones, including acetone, methylethyl ketone, 3-pentanone, and the like; esters, including methyl acetate, ethyl acetate, isopropyl acetate, methyl formate, ethyl formate, and the like; carboxylic acids, including formic acid, acetic acid, propionic acid, and the like; and ethers, including diethyl ether, dipropyl ether, dibutyl ether, diphenyl ether, diisopropyl ether, anisole, and the like. Aqueous solvents include water, or water with inorganic salts dissolved therein.

As used herein, the term “reacting” or “reaction” refers to the bringing together of designated chemical reactants such that a chemical transformation takes place generating a compound different from any initially introduced into the system.

In some embodiments, prior to product isolation, a reducing agent can be added to the reaction mixture to quench any remaining molecular iodine or any other source capable of iodine atom transfer. Those of skill in the art will recognize that the particular reducing reagent used will depend on the particular iodinating reagent used, the solvents used for reaction and production isolation, and the product of the reaction to be isolated. Preferred reducing agents include inorganic sulfur containing reagents such as group I or group II metal sulfites, bisulfites, and thiosufates. In some preferred embodiments, the reducing agent is sodium thiosulfate and/or SO₂(gas).

It will be appreciated by those of skill in the art that the reaction between the micronized amino-aryl compound and the iodinating reagent, depending on the identity of the iodinating reagent, can generate highly acidic byproducts such as hydrogen iodide. In some cases, it is possible that such by-products can be reactive with the reaction starting material, reaction product or both, and can result in a reduced yield of the desired product. Therefore, it is sometimes preferable to include a weak base to buffer any strongly acidic reaction byproducts, such as hydrogen iodide. Preferred bases include weak inorganic bases such as group I or II carbonates, bicarbonates, phosphates, hydrogen phosphates, and the like. Some preferred weak bases include NaHCO₃ and CaCO₃.

As used herein, the term “alkyl” or “alkylene” is meant to refer to a saturated hydrocarbon group that is straight-chained or branched. Example alkyl groups include methyl (Me), ethyl (Et), propyl (e.g., n-propyl and isopropyl), butyl (e.g., n-butyl, isobutyl, s-butyl, t-butyl), pentyl (e.g., n-pentyl, isopentyl, neopentyl) and the like. In some preferred embodiments, alkyl groups can contain from 1 to about 20, from 2 to about 20, from 1 to about 10, from 1 to about 8, from 1 to about 6, from 1 to about 4, or from 1 to about 3 carbon atoms.

As used herein, “halo” or “halogen” refers to fluoro, chloro, bromo, and iodo.

As used herein, “alkoxy” refers to an —O-alkyl group. Example alkoxy groups include methoxy, ethoxy, propoxy (e.g., n-propoxy and isopropoxy), t-butoxy, and the like.

In some embodiments, the compounds of Formula I can contain a nitrogen protecting group at position R₆ or R₇. Representative protecting groups can be found in, for example, Greene, T. W., and Wuts, P. G. M, Protective Groups In Organic Synthesis, 3^{rd} ed., John Wiley & Sons, NY, 1999, incorporated herein by reference.

At various places in the present specification substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl and C₆alkyl.

The reactants or products of the present invention can contain an asymmetric atom, and some of the compounds can contain one or more asymmetric atoms or centers, which can thus give rise to optical isomers (enantiomers) and diastereomers. The present invention includes such optical isomers (enantiomers) and diastereomers (geometric isomers); as well as the racemic and resolved, enantiomerically pure R and S stereoisomers; as well as other mixtures of the R and S stereoisomers and salts, including pharmaceutically acceptable salts, thereof. Optical isomers can be obtained in pure form by standard procedures known to those skilled in the art, including but not limited to diastereomeric salt formation, kinetic resolution, and asymmetric synthesis. It is also understood that this invention encompasses all possible regioisomers, and mixtures thereof, which can be obtained in pure form by standard separation procedures known to those skilled in the art, including but not limited to column chromatography, thin-layer chromatography, and high-performance liquid chromatography.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry, or by chromatography such as high performance liquid chromatography (HPLC) or thin layer chromatography.

The reactions of the processes described herein can be carried out at appropriate temperatures, which can be readily determined by the skilled artisan. Reaction temperatures will depend on, for example, the melting and boiling points of the reagents and solvent, if present; the thermodynamics of the reaction (e.g., vigorously exothermic reactions are typically carried out at reduced temperatures); and the kinetics of the reaction (e.g., a high activation energy barrier typically necessitates elevated temperatures). “Elevated temperature” refers to temperatures above room temperature (about 20° C.) and “reduced temperature” refers to temperatures below room temperature.

The reactions of the processes described herein can be carried out in air or under an inert atmosphere. Typically, reactions containing reagents or products that are substantially reactive with air can be carried out using air-sensitive synthetic techniques that are well known to the skilled artisan.

It is appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

The processes of this invention are suitable for the preparation of compounds of Formula I on any convenient scale, for example, greater than about 0.01 mg, 0.10 mg, 1 mg, 10 mg, 100 mg, 1 g, 10 g, 100 g, 1 kg, 10 kg or more. The processes are particularly advantageous for the large scale (e.g., greater than about ten gram) preparation of iodinated amino-aromatics.

The invention will be described in greater detail by way of a specific example. The following example is offered for illustrative purposes, and is not intended to limit the invention in any manner. Those of skill in the art will readily recognize a variety of noncritical parameters that can be changed or modified to yield essentially the same results.

### EXAMPLE 1

### Preparation of 4-chloro-2-iodoaniline

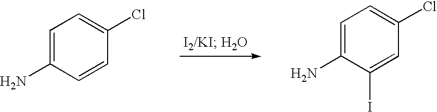

An aqueous solution of potassium iodide (50 g, 0.301 mol), iodine (76 g, 0.299 mol), and water (100 mL) is added over 45 min. to a stirred suspension of 4-chloroaniline (35 g, 0.276 mol, 50 um average particle size), sodium hydrogen carbonate (37 g, 0.440 mol) and water (210 mL). The mixture is stirred for 3-5 hours. The solid is collected by filtration and washed with water, then dried to provide 4-chloro-2-iodoaniline (69.6 g, 100% yield, 98.5% purity). ¹H NMR (CDCl3): δ 7.56 (d, 1H, J=2.4 Hz), 7.12 (dd, 1H, J=2.4 Hz, 8.6 Hz), 6.75 (d, 1H, J=8.6 Hz), 5.37 (br s, 2H).

As those skilled in the art will appreciate, numerous changes and modifications may be made to the preferred embodiments of the invention without departing from the spirit of the invention. It is intended that all such variations fall within the scope of the invention.

It is intended that each of the patents, applications, and printed publications, including books, mentioned in this patent document be hereby incorporated by reference in their entirety.

1. A process for the preparation of a compound of Formula I: 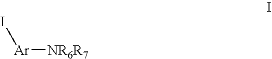
wherein:
Ar is a mono-, bi- or tricyclic aryl or heteroaryl ring system optionally containing up to four substituents independently selected from the group consisting of halogen, C₁₋₆ alkyl, CN, NO₂, CHO, COC₁₋₆ alkyl, CO₂H, CO₂C₁₋₆ alkyl, C₁₋₆ alkoxy, phenyl and C₁₋₆ thioalkyl, wherein the phenyl can be optionally substituted with from 1 to 3 substituents independently selected from the group consisting of C₁₋₃ alkyl, halogen, C₁₋₃ alkoxy, CN, NO₂, CHO, and phenyl;
R₆ and R₇ are each independently selected from the group consisting of H, C₁₋₆ alkyl and a nitrogen protecting group;
comprising:
reacting an amino aryl compound of Formula II: 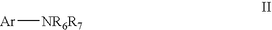
with an iodinating agent;
wherein the amino aryl compound of Formula II has an average particle size of about 100 μm or less.

2. The process of claim 1 wherein the amino aryl compound of Formula II is an aniline of Formula III: 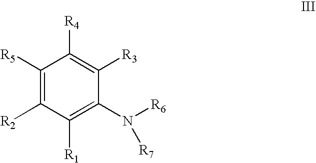
wherein R₅ is selected from the group consisting of halogen, C₁₋₆ alkyl, CN, NO₂, CHO, COC₁₋₆ alkyl, CO₂H, CO₂C₁₋₁₆ alkyl, C₁₋₆ alkoxy, phenyl and C₁₋₆ thioalkyl, wherein the phenyl can be optionally substituted with from 1 to 3 substituents selected from the group consisting of C₁₋₃ alkyl, halogen, C₁₋₃ alkoxy, CN, NO₂, CHO, and phenyl; and
R₁, R₂, R₃ and R₄ are each independently selected from the group consisting of hydrogen, halogen, C₁₋₆ alkyl, CN, NO₂, CHO, COC₁₋₆ alkyl, CO₂H, CO₂C₁₋₆ alkyl, C₁₋₆ alkoxy, C₁₋₆ thioalkyl and phenyl, wherein the phenyl can be optionally substituted with from 1 to 3 substituents selected from the group consisting of C₁₋₃ alkyl, halogen, C₁₋₃ alkoxy, CN, NO₂, CHO and phenyl;
provided that at least one of R₁ and R₃ is H.

3. The process of claim 2 wherein R₅ is halogen or C₁₋₆ alkyl; and R₁, R₂, R₃, R₄, R₆ and R₇ are each hydrogen.

4. The process of claim 3 wherein R₅ is halogen.

5. The process of claim 2 wherein the compound of Formula II has an average particle size of less than about 80 μm.

6. The process of claim 2 wherein the compound of Formula II has an average particle size of less than about 60 μm.

7. The process of claim 2 wherein the compound of Formula II has an average particle size of less than or equal to about 50 μm.

8. The process of claim 2 wherein the compound of Formula II has an average particle size of less than or equal to about 40 μm.

9. The process of claim 2 wherein the compound of Formula II has an average particle size of between about 30 μm and about 60 μm.

10. The process of claim 2 wherein the compound of Formula II has an average particle size of between about 40 μm and about 50 μm.

11. The process of claim 2 wherein the iodinating agent is molecular iodine.

12. The process of claim 2 wherein the reaction of the amino aryl compound of Formula II and the iodinating agent is performed in the presence of a group I or group II metal iodide.

13. The process of claim 12 wherein the metal iodide is potassium iodide.

14. The process of claim 11 wherein the reaction of the amino aryl compound of Formula II and the iodinating agent is performed in an aqueous solution.

15. The process of claim 14 wherein the aqueous solution is buffered with a weak base.

16. The process of claim 14 wherein the aqueous solution is buffered with NaHCO₃.

17. The process of claim 2 wherein the reaction of the amino aryl compound of Formula II and the iodinating agent is performed in an aqueous solution comprising molecular iodine, or a group I or group II metal iodide, or both molecular iodine and a group I or group II metal iodide.

18. The process of claim 17 wherein the metal iodide is potassium iodide.

19. The process of claim 18 wherein the aqueous solution is buffered with a weak base.

20. The process of claim 19 wherein the aqueous solution is buffered with NaHCO₃.

21. The process of claim 20 wherein the potassium iodide, molecular iodine, or both are added to a mixture of the amino aryl compound of Formula II, NaHCO₃ and water.

22. The process of claim 20 wherein the potassium iodide and molecular iodine are added as an aqueous solution to a mixture of the amino aryl compound of Formula II, NaHCO₃ and water.

23. The process of claim 17 wherein the molecular iodine is employed in an amount of from about 1 to about 1.5 equivalents relative to the amino aryl compound of Formula II.

24. The process of claim 17 further comprising the step of adding an inorganic reducing agent to the mixture subsequent to iodine addition and prior to product isolation.

25. The process of claim 24 wherein the inorganic reducing agent is a group I or II thiosulfate, a group I or II metal sulfite or a group I or II metal bisulfite.

26. The process of claim 25 wherein the inorganic reducing agent is sodium thiosulfate.

27. The process of claim 17 wherein the compound of Formula I is isolated by filtration.

28. The process of claim 27 wherein the filtered compound of Formula I is washed with a solvent.

29. The process of claim 28 wherein the filtered compound of Formula I is washed with water.

30. A process comprising the steps of:
(a) providing 4-chloroaniline in the form of particles having an average size of about 100 μm or less; and
(b) reacting the 4-chloroaniline with an iodinating agent under conditions effective to form 2-iodo-4-chloroaniline.

31. The process of claim 30 wherein the 4-chloroaniline particles have an average size of about 60 μm or less.

32. The process of claim 30 wherein the 4-chloroaniline particles have an average size of about 50 μm or less.

33. The process of claim 30 wherein the iodinating agent is molecular iodine.

34. The process of claim 33 wherein the reaction of the 4-chloroaniline and the molecular iodine is performed in an aqueous solution that further comprises a group I or group II metal iodide and a weak base buffer.

35. The process of claim 34 wherein the metal iodide is potassium iodide, and the weak base buffer is NaHCO₃.

36. The process of claim 35 wherein the potassium iodide and molecular iodine are added to a mixture of the 4-chloroaniline and the NaHCO₃ in water.

37. The process of claim 35 wherein the potassium iodide and molecular iodine are added as an aqueous solution to a mixture of the 4-chloroaniline and the NaHCO₃ in water.

38. The process of claim 35 wherein the molecular iodine is employed in an amount of from about 1 to about 1.5 equivalents relative to the amount of 4-chloroaniline.

39. The process of claim 35 further comprising the step of adding an inorganic reducing agent to the mixture subsequent to iodine addition and prior to product isolation.

40. The process of claim 39 wherein the inorganic reducing agent is sodium thiosulfate.

41. The process of claim 40 further comprising isolating the 2-iodo-4-chloroaniline by filtration.
